(19) United States
(12) Patent Application Publication
(10) Pub. No.: US 2020/0055150 A1
(43) Pub. Date: Feb. 20, 2020

(54) ULTRA HIGH VACUUM LASER WELDER SYSTEM

(71) Applicant: Medical Imaging Solutions USA, LLC, Woodstock, GA (US)

(72) Inventor: Farzad Sadeghi Tohidi, Woodstock, GA (US)

(21) Appl. No.: 16/545,756

(22) Filed: Aug. 20, 2019

Related U.S. Application Data
(60) Provisional application No. 62/719,827, filed on Aug. 20, 2018.

(51) Int. Cl. B23K 31/02; B23K 26/12; B23K 26/282; A61B 6/03; H01J 35/24

(52) U.S. Cl. CPC B23K 31/027; B23K 26/1224; B23K 2101/06; A61B 6/035; H01J 35/24; B23K 26/282

(57) ABSTRACT

Herein disclosed in a vacuum laser welding system which includes a chamber in which to generate a vacuum, a roughing pump and turbo pump for generating an ultra-high vacuum environment, temperature and pressure gauges to monitor the levels inside of the system, a cooling fan to reduce the temperature outside of the system, a laser viewport in order for the laser to penetrate without any deflection or leakage, a jig and fixture to mount the X-ray Tube insert inside of the system, and relief valves to release the pressures inside of the system.

### CROSS REFERENCE TO RELATED APPLICATIONS

This application is a non-provisional of and claims priority to U.S. Provisional Application Ser. No. 62/719,827, filed Aug. 20, 2018, pending, which application is hereby incorporated by this reference in its entirety for all purposes as if fully set forth herein.

### BACKGROUND

### Field of the Invention

Embodiments described herein relate to field of X-ray tube reclamation, and particularly, but not limited to, the field of computed tomography (CT) and X-ray radiography in order to extend the life of the X-ray tubes.

### Background

In a Computer Tomography (CT) device, an X-Ray tube generates radiation passing through the subject assigned for imaging and installed on the gantry for the purpose of revolution.

### SUMMARY

Accordingly, the present application is directed to a vacuum welding system that obviates one or more of the problems due to limitations and disadvantages of the related art.

An advantage of the present invention is to provide a vacuum laser welding system that allows extending the lives of X-Ray tubes of CT scanners and radiography devices by properly regenerating the vacuum inside of the insert without damaging/changing any prior components of the original design.

Additional features and advantages of the invention will be set forth in the description which follows, and in part will be apparent from the description, or may be learned by practice of the invention. The advantages will be realized and attained by the structure particularly pointed out in the written description and claims hereof as well as the appended drawings.

It is to be understood that both the foregoing general description and the following detailed description are exemplary and explanatory only, and are not restrictive of the invention as claimed.

### BRIEF DESCRIPTION OF THE DRAWINGS

The accompanying figures, which are incorporated herein and form part of the specification, illustrate an ultra-high vacuum welder system according to principles described herein. Together with the description, the figures further serve to explain the principles of the vacuum laser welding system described herein and thereby enable a person skilled in the pertinent art to make and use the described vacuum laser welding system.

FIG. 1A is a schematic illustration of an example X-Ray generation device of the type to be serviced according to principles described herein;

FIG. 1B is an illustration of another example X-Ray generation device of the type to be serviced according to principles described herein; and

FIG. 2 is a schematic illustration of an embodiment of the ultra-high vacuum welder system according to principles described herein;

### DETAILED DESCRIPTION

The following detailed description of example implementations refers to the accompanying drawings. The same reference numbers in different drawings may identify the same or similar elements.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

Implementations as provided herein provide a system and method of creating an ultra-high vacuum and welding back together an X-Ray Tube Assembly, and relate to methods and devices for repairing the X-ray tube.

Typically X-Ray tubes are sealed with a vacuum level of about 10⁻⁶ Torr and mounted inside a lead shielded radiation enclosure called a housing or casing. The housing is attachable to installable in or enclosable in the X-Ray scanner, typically, but not limited to, a Computer Tomography (CT), fluoroscopic, or X-Ray machine. The housing is filled with a fluid of synthetic or petroleum derivative, generally referred to as insulating oil. Over time the vacuum level slowly decreases and results in high voltage arcing, corona discharge, and other failure modes. Also, there is possibility of thermal fatigue related damages on both anode and cathode sides.

In a CT device, the X-Ray tube generates radiation passing through the subject assigned for imaging and installed on the gantry for the purpose of revolution. A detector assembly includes an array of detectors with purpose of changing radiation to electrical signals. A high voltage (HV) transformer is a device that alters the voltage of incoming electricity to a series of high voltage (HV) pulses needed for the X-Ray generation. There is a circular channel with a gaseous coolant between the stationary platform and the gantry, as well as a conduit with a circulating coolant exposed to heat generating components so that the generated heat can be eliminated from the system. The cooling procedure happens through a closed circulation system to avoid contaminating the environment around the imaging apparatus. The X-Ray source, or called here X-Ray tube, which is the heart of the X-Ray generating system, includes a radiation shielded housing, an X-Ray window, a central rotary part including anode and cathode, filament, ball bearings, a rotation source, heat and pressure sensors and electrical signal terminals.

The housing is substantially filled by an insulating liquid, generally a type of mineral or synthetic oil. The X-Ray tube assembly is attached to diagnostic medical imaging modalities such as computed tomography, radiography, fluoroscopy, mammography, catheter lab and angiography or the like. High level of vacuum is required inside the X-Ray tube insert to permit the electron flow from the cathode to the anode, which results in X-Ray generation. During the X-Ray tube operation, thermal shock could result in crack initiation and propagation at ceramic isolator of cathode and leads to vacuum leakage over time which introduces gases to the system and malfunction of system. In other words, the contamination inside the X-Ray tube insert results in lowering the vacuum level and leads to arcing which in turn leads to the failure of the tube and the necessity of regeneration of high vacuum level inside the insert.

A method described herein regenerates the vacuum inside of the X-Ray tube insert with or without replacing the parts inside the insert to extend the useful life of the system without requiring changing components or materials of the original design.

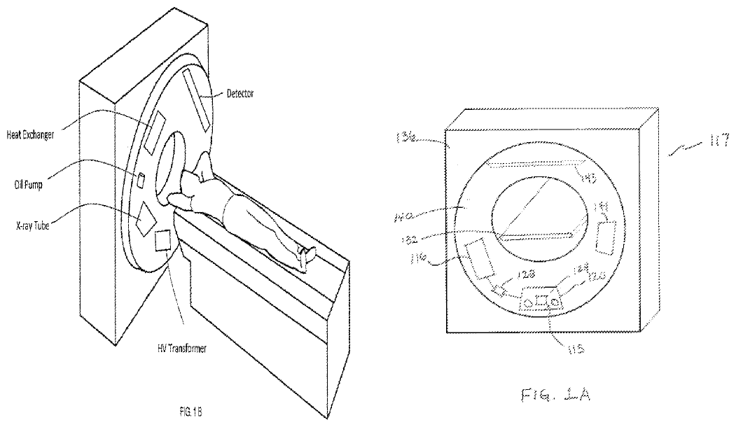

As illustrated in an exemplary CT device 117 in FIG. 1, an X-Ray tube assembly generally includes an X-Ray tube, the housing 120 including insulating oil 124, a heat exchanger 116 for removing heat from the insulating oil and pump 128 for circulating insulating oil.

For most X-Ray products, the end of life is primarily predicated by an arcing process. Arcing is a particular problem for CT machines, and when the arcing increases to the point that the machine cannot no longer function, the machine must be removed from patient use. Repair typically requires de-installation of the X-Ray tube assembly partially or in its entirety, including the X-Ray tube, the housing including insulating oil, the pump and the heat exchanger, and associated lead shielding.

According to principles described herein, the X-Ray tube insert can be removed from the CT machine. Servicing the X-Ray tube may require that the X-Ray Tube be welded such that the vacuum within the X-Ray Tube must be maintained.

The purpose of this system described is to weld together a cut X-ray tube insert after an ultra-high vacuum between 10⁻⁷ to 10⁻⁹ Torr has been regenerated in the tube so that the vacuum can be achieved and maintained with a high degree of accuracy. Through the use of electrical/rare magnets, the laser weld performed under this level of vacuum is possible. The magnets are designed and placed in the system in order to trap the evaporation of molecules, which has an adverse effect on the level in the chamber.

The vacuum laser welding system may include a chamber that can attain 10⁻⁷ Torr and higher levels of vacuum. A laser with appropriate power for laser welding the material of the insert is provided. A roughing pump may be used to generate a vacuum inside the chamber. A turbo pump may be used to accelerate the roughing pump's power and speed in order to obtain greater vacuum levels. Magnets in the chamber are used to trap evaporating molecules from the laser weld. At least one heater is provided to cover connection points on the system in order to direct the flow of molecules and reduce leakage. A laser viewport is used for the laser module to pass the laser weld through without any deflection and/or leakage. A wire feedthrough compatible with ultra-high vacuum is provided to heat up the cathode and measure the temperature inside the insert. At least one jig and fixture to precisely align and hold the insert components supposed to be weld together. An automatic wire feeder system provides am appropriate amount of material as the weld filler. The roughing pump is connected to the turbo pump, the turbo pump is connected to the chamber, and all other connections to the chamber are closed in order to generate vacuum levels at least 10⁻⁷ Torr.

In general, a system that can be loaded/unloaded with an X-Ray tube insert for welding, generate an ultra-high vacuum, and weld back together the insert with precision while measuring/observing all vacuum levels and temperatures is provided.

Rare earth magnets or electrical magnets are placed closely to the weld line to trap escaping molecules when the laser comes into contact with the insert (or other object being welded) and begins the weld, allowing the system to maintain a vacuum level of at least 10⁻⁷ Torr.

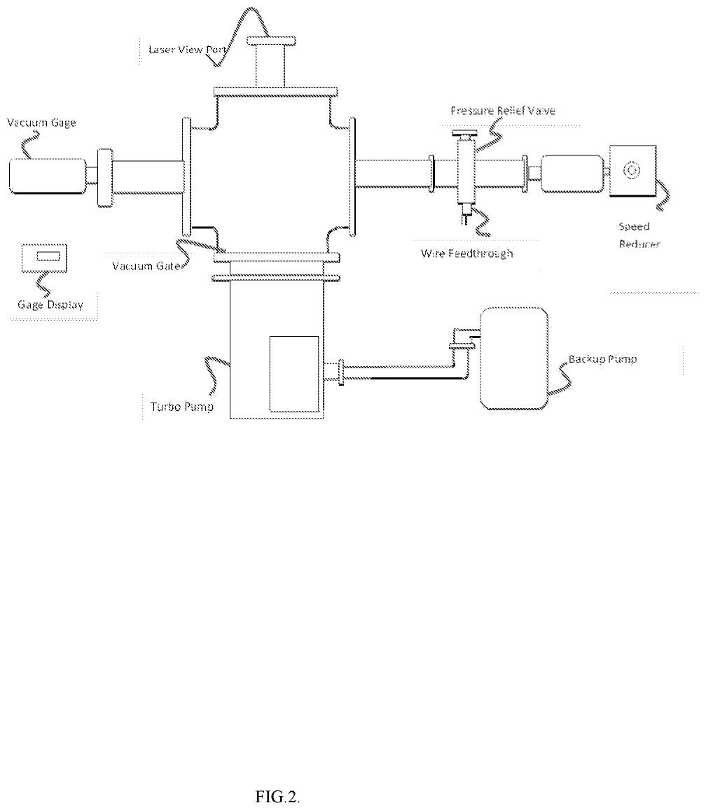

As illustrated in FIG. 2, a vacuum laser welding system according to principles described herein may include a vacuum chamber, a roughing pump (not shown) to generate the vacuum, a turbo pump to accelerate the roughing pump and attain ultra-high vacuum levels, a vacuum/pressure gauge to observe the level of vacuum, a motor (not shown) to rotate the X-Ray tube insert, a speed/gear-box reducer to slow the motor speed, heaters (not shown) to direct molecules away from openings, a cooling fan (not shown) to reduce temp of system, a laser viewport window to allow visual access to the system to allow for laser welding inside of the system, temperature gauges (not shown) to monitor the temperature of the heaters and the system, a jig and fixture (not shown) to mount the insert inside of the vacuum chamber, relief valves (not shown) in order to release/control the vacuum level, a mobile platform (not shown) that allows the vacuum laser system to be transported to other facilities, a laser (not shown) to weld the insert, magnets (not shown) to trap the evaporation of molecules from the laser weld, and a control panel (not shown) to control the heats, pumps, and lights in/on the system. According to principles described herein, a vacuum laser welding system for regenerating and maintaining a vacuum inside the insert of X-Ray tube assembly during welding includes a vacuum chamber than can a chamber that can attain 10⁻⁷ Torr and higher levels of vacuum. The parts to be welded may be placed in the vacuum chamber. While described herein as a vacuum chamber, in some circumstances, an inert gas may be used in place of the vacuum in the chamber. Principles for laser welding as described herein, may be applicable. The system further includes a laser that offers appropriate power for welding the insert.

The vacuum may be generated and maintained by at least one roughing pump to generate vacuum inside the chamber and at least one turbo pump to accelerate the roughing pump's power and speed in order to obtain greater vacuum levels. For example, the roughing pump may be connected to the turbo pump; the turbo pump may be connected to the chamber; and all other connections to the chamber may be closed in order to generate vacuum levels at least 10⁻⁷ Torr.

Magnets are provided in the vacuum chamber. The magnets may be rare earth magnets or electrical magnets. The magnets may be placed close to the weld line to trap escaping molecules when the laser comes into contact with the part to be welded and begins the weld. The magnets trap molecules that evaporate as a result of the laser welding process, thus allowing a vacuum to be maintained without influence of the escaping molecules

A heater may be provided to cover connection points on the system in order to direct the flow of molecules and reduce leakage. A laser viewport may be provided for the laser module to pass through without any deflection and/or leakage. A wire feedthrough compatible with ultra-high vacuum may be provided to heat up the cathode and measure the temperature inside the insert. A jig and fixture may be provided in the vacuum chamber for receiving the part to be welded and to align and hold the components that are to be weld together. An automatic wire feeder system may be included to provide a right amount of material as the weld filler.

The chamer may be a four-way cross chamber, for example, and the connections thereto may be covered with flexible heaters to direct the flow of molecules and reduce any vacuum leakage from the system. Magnets are placed closely in relation to the weld line to trap any evaporating molecules. A control panel may be wired to power the roughing pump, turbo pump, strip heaters, and lights. The control panel may use LED switches and may be equipped with an emergency stop to shut down all components. The entire system including the electronics may be mounted on a six-wheel platform to hold all components in place and facilitate transport.

In general, a system that can be loaded/unloaded with X-Ray tube inserts, generate an ultra-high vacuum, and precisely weld back together the insert while measuring/observing all vacuum levels and temperatures.

An example implementation of the system described herein may include placing the part(s) to be welded in the jig within the vacuum chamber. The vacuum chamber is sealed and a vacuum created within the chamber and the part(s) to be welded. The part(s) is/are welded appropriately. During the welding process, the magnets capture the particles/molecules generated by the welding process (escaping from the part during the weld). If the magnets are electromagnets, such magnets should be energized before the welding process begins. After the welding process is completed, the electromagnets may be de-energized.

Herein disclosed in a vacuum laser welding system which includes a chamber in which to generate a vacuum, a roughing pump and turbo pump for generating an ultra-high vacuum environment, temperature and pressure gauges to monitor the levels inside of the system, a cooling fan to reduce the temperature outside of the system, a laser viewport in order for the laser to penetrate without any deflection or leakage, a jig and fixture to mount the X-ray Tube insert inside of the system, and relief valves to release the pressures inside of the system.

As described herein, the system and method of ultra-high vacuum welding is not limited to application to an X-ray Tube cylinder or insert and may have broader application, as contemplated herein.

While various embodiments have been described above, it should be understood that they have been presented by way of example only, and not limitation. It will be apparent to persons skilled in the relevant art that various changes in form and detail can be made therein without departing from the spirit and scope of the present invention. Thus, the breadth and scope of the present invention should not be limited by any of the above-described exemplary embodiments, but should be defined only in accordance with the following claims and their equivalents.

What is claimed is:

1. A laser welding system, comprising:
a chamber that can attain at least 10⁻⁷ Torr;
a laser that offers appropriate power for welding the insert; and
magnets in the chamber in order to trap evaporating molecules from the laser weld

2. The system of claim 1, further comprising:
at least one roughing pump to generate vacuum inside of the chamber; and
at least one turbo pump wherein the roughing pump is connected to the turbo pump and the turbo pump is connected to the chamber.

3. The system of claim 1, wherein the magnets comprise rare earth magnets.

4. The system of claim 1, wherein the magnets comprise electrical magnets.

5. The system of claim 1, wherein the magnets are placed adjacent to the weld line.

6. The system of claim 3, wherein the magnets are placed adjacent to the weld line.

7. The system of claim 4, wherein the magnets are placed adjacent to the weld line.

8. The system of claim 2, wherein all other connections to the chamber are closed in order to generate vacuum levels at least 10⁻⁷ Torr.

9. The system of claim 1, further comprising:
at least one heater to cover connection points on the system in order to direct the flow of molecules and reduce leakage;

10. The system of claim 1, further comprising:
a laser viewport for the laser module to pass through without any deflection and/or leakage;

11. The system of claim 1, further comprising:
a wire feedthrough compatible with ultra-high vacuum to heat up the cathode and measure the temperature inside the insert

12. The system of claim 1, further comprising:
at least one jig and fixture to align and hold the insert components during welding.

13. The system of claim 1, further comprising:
an automatic wire feeder system to provide weld filler during a welding process.

14. A method of conducting a weld in a vacuum comprising:
placing an item to be welded into a vacuum chamber having at least one magnet therein;
sealing the vacuum chamber;
creating a vacuum in the chamber; and
welding the item, the magnets attracting particles dispersed during welding the item.
